(19) United States
(12) Patent Application Publication
(10) Pub. No.: US 2003/0049852 A1
(43) Pub. Date: Mar. 13, 2003

(54) Chemical spot test for lead in paint and other media

(76) Inventor: Sandra Lynn Cole, Dallas, TX (US)

Correspondence Address: CARSTENS YEE & CAHOON, LLP, P O BOX 802334, DALLAS, TX, 75380

(21) Appl. No.: 10/217,037

(22) Filed: Aug. 12, 2002

Related U.S. Application Data
(63) Continuation of application No. 09/336,614, filed on Jun. 18, 1999, now Pat. No. 6,489,170.

(51) Int. Cl. G01N 33/20

(52) U.S. Cl. 436/77

(57) ABSTRACT

A one-step chemical spot test for the qualitative determination of lead in lead-based paint and other media. This test is based upon the reaction of lead with sodium rhodizonate under strong acid conditions to develop an intense purple colored complex.

### BACKGROUND OF THE INVENTION

1. Technical Field

This application is a Continuation of U.S. application Ser. No. 09/336,614, filed on Jun. 18, 1999. The present invention relates to a method for qualitatively determining the presence of lead in lead-based paint and other media while avoiding barium interferences by use of sodium rhodizonate and a strong acid.

2. Description of Related Art

For hundreds of years, people have used lead for different purposes. Lead was used by the Romans for soldering pipes, by others in crystal glassware, and currently in paint mixtures, as well as other applications. The hazards of lead poisoning have been known, but only in relatively recent times has the extent of the threat to children moved to the forefront.

The ingestion of lead is harmful to people of all ages, but is more damaging to children under six, and unborn fetuses. The developing nervous system is more susceptible to the toxic effects of the lead. Children who have been exposed to lead exhibit behavioral and cognitive impairment at low levels, with higher levels resulting in anemia, brain damage and other irreversible effects. The risk of lead poisoning to children from lead-based paint was identified as early as 1897.

Children are exposed to lead in lead-based paint through normal childhood behaviors, such as sucking and chewing on painted surfaces, and ingesting paint chips from damaged areas. If lead-based paint is removed without appropriate precautions, the airborne particles permeate the area, and can be ingested or inhaled by both children and adults.

It is believed that ninety percent of houses built before 1940 contain lead paint. In houses built before 1950, paints used in housing contained as much as 50% lead by dry weight. After the 1940s, the use of lead-based paint decreased in residential homes.

It is estimated that more than 70% of homes built before 1980 have lead in paint and fixtures. It was commonly used in areas where durability was desired, such as trim, cabinets, and outdoor areas.

In 1972 the Consumer Products Safety Commission made the first effort to regulate the lead content in paint. The Commission established a maximum lead content in paint at 0.5% lead w/w in residential paint. This limit was considered to be “safe”. In 1977, lead was even further restricted from use in residential paints due to the risk of lead poisoning in children. Any lead content below 0.06% was considered as “lead-free” paint. Any paint with a lead content greater than 0.06% was still considered to be lead-based paint.

In 1990, the U.S. Department of Housing and Urban Development (“HUD”) published “Lead-Based Paint: Interim Guidelines for the Identification and Abatement of Lead-Based Paint in Public and Indian Housing.” The HUD guidelines described technical protocols, practices and procedures for testing, abatement, and worker protection in cleanup and disposal of lead-based paint. The HUD guidelines also required inspection of public and Indian housing before 1994, and abatement if the amounts exceeded an action level of 0.5% lead w/w, or 1 mg/cm² mass/area concentration.

Although there are no federal requirements to abate lead in private housing, in 1992 the Residential Lead-Based Hazard Reduction Act, Title X, was passed, to become effective in 1995. Title X established new requirements for homeowners and Federal agencies, and new actions to improve the safety and effectiveness of lead-based paint identification and remediation activities. This act requires the sellers of homes to disclose the existence of any lead-based paint or hazard in pre-1978 homes, and allow purchasers 10 days to inspect before becoming obligated to purchase the house.

In response to requirements made by Title X, HUD issued new guidelines, entitled “The Guidelines for the Evaluation and Control of Lead-Based Paint Hazards in Housing.” This document provides detailed guidance on identifying lead-based paint and associated hazards in housing, and controlling the hazards safely and efficiently. A significant change made by Title X and the subsequent guidelines was in the working definition of lead-based paint. Lead-based paint hazards now became “any condition that causes exposure to lead from lead-contaminated dust; bare lead-contaminated soil; or lead-based paint that is deteriorated or intact lead-based paint present on surfaces, or impact surfaces that would result in adverse human health effects.”U.S. Department of Housing and Urban Development, The Guidelines for the Evaluation and Control of Lead-Based Paint Hazards in Housing, Government Printing Office, 1995, p. 1-8. Under this definition, intact lead-based paint was not considered a hazard, but should be monitored and controlled. An exception to monitoring plans was still made for Indian and public housing, where the requirement exists to abate if the housing is modernized.

Identifying lead-based paint by HUD guidelines can be accomplished by either portable x-ray fluorescence analyzers (XRF) or by laboratory analysis of paint chips. XRFs are expensive to purchase, have radioactive sources, and operators must be trained and licensed. A laboratory analysis is time-consuming, and may also be very costly. Since lead-based paint hazards have gained attention, less costly methods have been developed to identify qualitatively lead-based paint. Two tests that have been developed include sodium sulfide, and a one-step sodium rhodizonate test. These tests have not gained a wide acceptance due to some of their limitations. The limitations include false positives, false negatives, and difficulties seeing the appropriate color change indicating a positive result.

The “one-step red” sodium rhodizonate test is actually the first step of a test which has been used in the past for the identification of both barium and lead. Use of the “one-step red” test ignores the previously established limitations of the same procedure. In the past, the results provided by the red color in a positive “one-step red” test indicated the presence of both lead and barium. An additional step was required to differentiate between the two, and for the results to be conclusively interpreted as lead.

Recent years have seen an application of a portion of the sodium rhodizonate test to a new area of interest in lead determination. With the concern regarding the presence of lead in paints used in the past, simple testing methods have become advantageous for use in the field. These tests allow the user to make a qualitative analysis of the lead content in a painted surface. The test results can provide the basis for determining the hazards which may arise from the paint removal, or continued exposure to the painted surface. If a field test is not available, the only alternative is instrumental analysis methods, which require laboratory testing or expensive field instruments. Simple testing kits, using the first step in the sodium rhodizonate test, were patented in the early 1990s. These became commercially available, and were accepted for qualitative lead identification in the field. The results of these tests were often used to decide the hazards of the painted surface and the method for paint removal. If the test indicated that the paint was lead-based, considerable expense could be incurred in the removal. Due to variations in the size and conditions of the building, local labor, the market competition, and the type of control selected, cost estimates are difficult, however, in 1991 HUD estimated that control in more than half of the affected housing units could be accomplished for less than $2500 apiece. This cost does not include the initial testing costs, a risk assessment if needed, or the relocation of the occupants during the hazard control.

The interpretation of the results obtained by the new methods in use, however, are in opposition to the interpretation currently accepted by the forensic science community.

In 1995, the American Society for Testing and Materials (ASTM) issued the “Standard Practice for Use of Qualitative Chemical Spot Test Kits for Detection of Lead in Dry Paint Films.” The methods used in this standard are the methods used by the commercial kits. Two types of chemical spot tests were evaluated for use in determining lead-based paint. One test uses sodium sulfide, and the other test uses sodium rhodizonate.

The first test uses the reaction of a clear sodium sulfide and lead in situ, or on paint chips. A positive reaction is indicated by a black or gray color. The advantage of this test is that it is easy, relatively inexpensive, and fast. One of the disadvantages is the potential generation of hydrogen sulfide, a toxic gas. Another disadvantage is the fact that the sulfide test is not specific for lead. Several other metal ions react to produce a black color, including iron, nickel, cobalt, copper, mercury and molybdenum. Additionally, a positive test may be difficult to see on a dark surface.

The second test described, and accepted, is the sodium rhodizonate test. The method described by the standard, however, is only the first step of the two-step test that has been used since the 1940s. The standard describes the test as the reaction of the sodium rhodizonate solution and lead to produce a pink or red complex under acid conditions. The ASTM standard further states that one of the advantages of the rhodizonate test is that “Under acid conditions, only lead reacts with the yellow/orange rhodizonate solution to give a pink to red color” and that the reaction with barium produces an orange color (ASTM, 1995a). The ASTM standard does not specify the pH of the acid conditions, however the method utilizes weak acid at a pH˜2.8.

There are several commercially available test kits which use the “one-step-red” sodium rhodizonate test as described in the ASTM method. Some of these include kits marketed under the names LeadCheck, Lead Alert, and EM Lead Test.

The LeadCheck test uses sodium rhodizonate and a weak acid applied directly to the painted surface. The Manufacturer's Information Sheet included with the test specifies that the appearance of pink, red-pink or red indicates the presence of lead. The Information Sheet states that “Since lead is the only substance that reacts with the dye to give the characteristic pink color, false positive reactions are not likely to occur.” The information sheet further identifies barium as the only other substance to react with sodium rhodizonate, however, the result is described as orange instead of pink.

These tests (the “one-step red” tests) fail to explain the inconsistencies of the positive results of these tests, with the interpretation of positive results considered to be conclusive by other researchers. The “One-Step Red” Sodium Rhodizonate Test excepts a red results as specifc for lead alone, where other researchers believe that this indicates the presence of both barium and lead.

The primary limitation of the “one-step red” process utilized by currently available test kits appears to be the lack of evidence for the premise that the red color indicates lead, and only lead. This assumption appears to be contrary to previously accepted studies. In the studies reported by the Defense Technical Information Center, the basic premise for the ASTM Spot Test Method, and a product evaluation produced by the OSHA Technical Center, all make the basic assumption that red or pink is conclusive for only lead, and reports that barium produces an orange complex. The OSHA document does, however, specify that “A positive test is evidence of the presence of lead or a positive interference.”

The presence of barium in the paint is largely neglected, and considered only as a possible interference. In the past, as well as currently, barium compounds are commonly used in various paint preparations. Barium acetate is used in paint and varnish driers, barium bromate is used as a corrosion inhibitor, and barium chloride is used in pigments. Barium chromate is used as a corrosion inhibitor as well as a pigment in paint. Barium citrate is a stabilizer for latex paints, and barium manganate is used as a paint pigment. Other barium compounds used in paints and as corrosion inhibitors include barium molybdate, barium nitrate, barium potassium chromate, barium sulfate, and barium zirconium silicate.

It is possible that the tests available are reacting to form a red complex, which indicates barium or lead, when the element present is only barium. This is a common ingredient of paint, which would result in a false positive. This response could certainly lead to unnecessary abatement measures.

An additional limitation is the accuracy rate of the “one-step red” test. Studies have indicated that the test is not accurate for lead 100% of the time. The study published by the Defense Technical Information Center (DTIC) reported a detection level of 0.38% w/w with a 95% probability of detection. The DTIC also reported an unacceptable level of false positives below 0.4%. These positives may have been due to the presence of barium, resulting in the red barium complex described by other researchers. The testing method used by the OSHA Technical Center appeared to have been limited to only the number of tests possible with each commercially available kit. The kits generally supply materials for one or two tests, so no accuracy rate was reported.

In the “one-step red” test, another limiting factor is the time required to react with lead chromate, a common ingredient of lead-based paint. The ASTM method specifies that if lead chromate is suspected, a negative result should be examined as long as 24 hours after testing, due to the slow reaction of the chromate with the test kit chemicals. The Manufacturer's Information Sheet from LeadCheck indicates that a positive result may still develop, from an apparent negative sample, anywhere from one hour to overnight.

Accordingly, a need exists for a relatively simple “one-step” method of detecting lead in a lead based paint or other media that is easy to administer, safe, and inexpensive. Preferably, this method should qualitatively identify the presence of lead without interference from other elements or compounds, such as barium, that might lead to a false positive indication. The method should also be highly accurate at relatively low detection levels and provide quick results within minutes of performing the test on a sample.

### SUMMARY OF THE INVENTION

A method for qualitatively detecting lead in lead based paint and other media is disclosed using sodium rhodizonate and hydrochloric acid in the presence of lead to form a blue/purple complex. This acidified complex has been accepted as unique to lead and is referred to as the “one-step purple” test.

The development of a “one-step purple” test fulfills a need for an inexpensive, reliable field test to be used for the qualitative determination of lead in paint and other media. This test avoids the conflict with the red barium-sodium rhodizonate complex identified in previous methods and safely provides quick and accurate results at relatively low lead concentrations.

The above as well as additional features and advantages of the present invention will become apparent in the following written detailed description.

### BRIEF DESCRIPTION OF THE DRAWINGS

The novel features believed characteristic of the invention are set forth in the appended claims and claims to be appended in the non-provisional filing of this application. The invention itself, however, as well as a preferred mode of use, further objectives and advantages thereof, will be best understood by reference to the following detailed description of illustrative embodiments when read in conjunction with the accompanying drawings, wherein:

FIG. 1 illustrates a flow chart of the general method of the invention.

### DETAILED DESCRIPTION

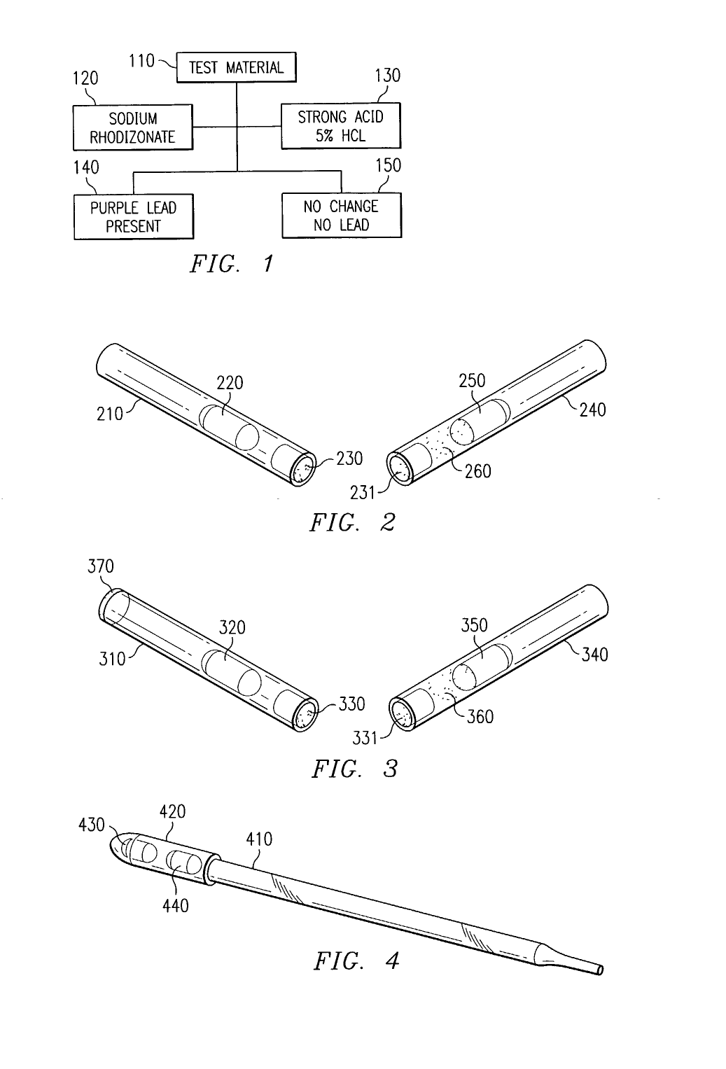

FIG. 1 shows a flow chart demonstrating the overall method of the invention. A test material 10 is first identified. This test material 10 could be a chip of paint suspected of containing lead or some other media (emulsion, slurry, liquid mixture, etc. . . . ) which requires testing to determine the presence of lead or a lead based compound. Sodium rhodizonate 20 and hydrochloric acid 50 are then applied to the test material 10 simultaneously or within a short period of time (thus a “one-step” test is performed). If after a period of time, for example a few minutes, the tested sample exhibits a purple tint 40 then lead or a lead-based compound has been detected. However, of there is no change 50 exhibited by the tested sample, then there is no lead or lead based compound in the material.

One preferred embodiment of the invention's method consists of applying hydrochloric acid to a cotton swab, scrubbing a painted surface in an area approximately 1 square centimeter in size, and applying one drop of sodium rhodizonate solution to the swab. Three drops of hydrochloric acid are considered optimum. Less than three drops does not adequately break down the paint matrix. The duration of scrubbing depends upon the paint deposit upon the swab. When paint is visible upon the swab, the scrubbing is stopped. If no paint is visible, the board is scrubbed for 5 seconds. One drop of sodium rhodizonate is then applied directly to the cotton swab. The presence of lead is indicated by a color change to purple/blue.

The solution of sodium rhodizonate prepared to the color of strong tea contributes to the best results. The hydrochloric acid recommended is a 5% solution, as this is the lowest concentration that gives accurate results.

Using the above disclosed method, the presence of lead has been detected in the latex-based based paint in the full range of variables. Tests using this method were positive in detecting from 40% to 0.016% lead acetate concentrations (w/w), with various solutions of sodium rhodizonate and various concentrations of hydrochloric acid. In oil-based paints, positive results were achieved from 40% to 0.016% lead concentrations, again using various combinations of hydrochloric acid with a 970 ppb sodium rhodizonate solution. The combination of 50% hydrochloric acid with 388 ppb solution resulted in a trace of purple/blue color, however the trace was not sufficient to be considered conclusive.

This “one-step purple” test is a substantial improvement in the art due to its accuracy, imperviousness to interference and false positive indications, and relatively fast reaction times. In short, this invention is safe, effective, and inexpensive, thus providing a superior testing means over those test methods presently enjoying considerable market success.

Additional information regarding various embodiments of the invention and descriptive detail can be found in Applicant's thesis on the invention attached hereto as appendix A, which is incorporated by reference for all purposes in support of the disclosure of Applicant's invention.

While the invention has been particularly shown and described with reference to preferred embodiment, it will be understood by those skilled in the art that various changes in form and detail may be made therein without departing from the spirit and scope of the invention.

What is claimed is:

1. A method of detecting lead in a media which comprises the steps of:
(a) exposing the media to a hydrochloric acid;
(b) exposing the media to sodium rhidozonate; and,
(c) monitoring the media for a visible color change.

2. The method of claim 1 wherein the color change of step (c) is a purple to blue tint.

3. The method of claim 1 wherein the rhodizonate is formed from a rhodizonic acid salt and water.

4. The method of claim 1 wherein the rhodizonate is formed from rhodizonic acid, disodium salt, and water.

5. The method of claim 1 wherein the rhodizonate concentration appears as a yellow/brown color.

6. The method of claim 1 wherein the dilute hydrochloric acid comprises a concentration of approximately 5%. volume to volume.

7. The method of claim 1 further comprising the steps of:
(d) comparing the color change to a color chart.

8. The method of claim 7 wherein the color chart contains a range of colors indicating the positive or negative detection of lead.

9. The method of claim 7 wherein the color chart contains a range of colors in which the positive colors are correlated to a detected lead concentration.

10. A method of detecting lead in a media which comprises the steps of:
(a) abrading the media;
(b) exposing the media to a hydrochloric acid;
(c) exposing the media to sodium rhidozonate; and,
(d) monitoring the media for a visible color change.

11. The method of claim 10 wherein the color change of step (d) is a purple to blue tint.

12. The method of claim 10 wherein the rhodizonate is formed from a rhodizonic acid salt and water.

13. The method of claim 10 wherein the rhodizonate is formed from rhodizonic acid, disodium salt, and distilled water.

14. The method of claim 10 wherein the sodium rhodizonate concentration appears as a yellow/brown color.

15. The method of claim 10 wherein the dilute hydrochloric acid comprises a concentration of approximately 5%. volume to volume.

16. The method of claim 10 wherein the color change of step (d) is further compared to a color chart.

17. The method of claim 16 wherein the color chart contains a range of colors indicating the positive or negative detection of lead.

18. The method of claim 16 wherein the color chart contains a range of colors in which the positive colors are correlated to a detected lead concentration.
